(19) United States
(12) Patent Application Publication
(10) Pub. No.: US 2026/0263085 A1
(43) Pub. Date: Sep. 10, 2026

(54) FAILSAFE TOURNIQUET

(71) Applicant: Tal MOSHKOVITZ, Pardes Hanna-Karkur (IL)

(72) Inventor: Tal MOSHKOVITZ, Pardes Hanna-Karkur (IL)

(21) Appl. No.: 19/657,605

(22) Filed: Apr. 24, 2026

Related U.S. Application Data
(63) Continuation-in-part of application No. PCT/IL2024/050981, filed on Oct. 7, 2024.

(30) Foreign Application Priority Data
Oct. 30, 2023 (IL) 308145

(51) Int. Cl. A61B 17/135

(52) U.S. Cl. CPC A61B 17/135

(57) ABSTRACT

Failsafe tourniquet including an initially elongated flat cuff having a cuff buckle end, a cuff free end, and a fastening arrangement for fastening a cuff section around a patient's limb for circumferentially applying a limb occlusion pressure. For tightening the cuff section encircled around a patient's limb, a user either operates a pneumatic device for inflating the cuff section or winds the pneumatic device in a windlass rod-like manner.

### FIELD OF THE INVENTION

The invention relates to medical equipment to limit or stop bleeding in general and medical equipment to limit or stop bleeding of a patient's limb.

### BACKGROUND OF THE INVENTION

There are different types of medical equipment including a cuff for encircling a patient's limb for circumferentially applying Limb Occlusion Pressure (LOP) to limit or stop bleeding. One medical equipment is a windlass tourniquet including a windlass rod for tightening a cuff around a patient's limb. Another medical equipment is a pneumatic tourniquet including an inflatable cuff. Exemplary medical equipment are described in inter alia CN 113316425, CN 209405087, U.S. Pat. Nos. 7,842,067, 7,892,253, 8,888,807, 10,136,900, 12,144,509, US 2015/0032149, US 2015/0257767, US 2021/0236141, US 2023/0270448, WO 2006/116413, and the like.

### SUMMARY OF THE INVENTION

The present invention relates to failsafe tourniquets for circumferentially applying a limb occlusion pressure to a patient's limb. The failsafe tourniquet includes an initially flat, elongated cuff having a cuff buckle end and a cuff free end and being configured for a cuff section to be encircled around a patient's limb and fastened therearound. The failsafe tourniquet includes an initially uninflated strip balloon deployed inside the cuff and a windlass strap integral with the cuff. The windlass strap includes a windlass strap section freely overlying the cuff's topside ply and deployed towards the cuff buckle end. The failsafe tourniquet includes a hand operated pneumatic device mounted on the windlass strap section. The hand operated pneumatic device includes an initially sealed gas cylinder containing compressed gas for selectively inflating the initially uninflated strip balloon to a predetermined limb occlusion pressure.

A user can tighten the cuff section encircled and fastened around a patient's limb either by operating the pneumatic device for puncturing the initially sealed gas cylinder for inflating the cuff section or winding the pneumatic device around the windlass strap section in a windlass rod-like manner similar to a windlass tourniquet. Operating the pneumatic device to inflate the cuff section to apply the strip balloon's predetermined limb occlusion pressure is typically quicker than using the failsafe tourniquet as a windlass tourniquet. If, however, it transpires the strip balloon's predetermined limb occlusion pressure is insufficient to stop bleeding, the user can wind the pneumatic device around the windlass strap section in a windlass rod-like manner similar to a windlass tourniquet to increase limb occlusion pressure. In an instance a user selects to initially use the failsafe tourniquet as a windlass tourniquet, the user can circumferentially apply a limb occlusion pressure smaller than, equal to or greater than the strip balloon's predetermined limb occlusion pressure.

Failsafe tourniquets of the present invention can be supplied in different cuff lengths and different lengths of cuff sections. Failsafe tourniquets of the present invention can be supplied with strip balloons for applying different pre-determined limb occlusion pressures. Failsafe tourniquets can be color coded or otherwise marked for indicating cuff lengths and/or lengths of cuff sections and/or predetermined limb occlusion pressures.

### BRIEF DESCRIPTION OF DRAWINGS

In order to understand the present invention and to see how it can be carried out in practice, preferred embodiments will now be described, by way of non-limiting examples only, with reference to the accompanying drawings, in which similar parts are likewise numbered, and in which:

FIG. 1 is a front leading pictorial view of a failsafe tourniquet including an initially flat, elongated cuff and a hand operated pneumatic device in an initial closed pocketknife state in accordance with one embodiment of the present invention.

FIG. 2 is a front leading pictorial view of the pneumatic device in its initial closed pocketknife state.

FIG. 3 is a rear trailing pictorial view of the pneumatic device in its initial closed pocketknife state.

FIG. 4 is a front leading pictorial view of the failsafe tourniquet including an initially flat, elongated cuff and its pneumatic device in a final open pocketknife state.

FIG. 5 is a partial cross section of the pneumatic device along line 5-5 in FIG. 4.

FIG. 6 is a front leading pictorial view of the pneumatic device in its final open pocketknife state.

FIG. 7 a rear trailing pictorial view of the pneumatic device in its final open pocketknife state.

FIG. 8 is a longitudinal cross section view of the failsafe tourniquet along line 8-8 in FIG. 1.

FIG. 9 is a longitudinal cross section view of the failsafe tourniquet along line 9-9 in FIG. 4.

FIG. 10A is a partial exploded view of the pneumatic device in its final open pocketknife state

FIG. 10B is a front leading pictorial view of a strip balloon and a windlass strap.

FIG. 11 is a front pictorial view of a failsafe tourniquet including an initially flat, elongated cuff and a hand operated pneumatic device in an initial closed pocketknife state in accordance with another embodiment of the present invention.

FIG. 12 is a schematic view showing use of the FIG. 1 failsafe tourniquet with an inflated cuff for circumferentially applying its strip balloon's predetermined limb occlusion pressure to a patient's limb.

FIG. 13 is a schematic view showing use of the FIG. 1 failsafe tourniquet with its pneumatic device in its final open pocketknife state as a windlass tourniquet for circumferentially applying limb occlusion pressure to a patient's limb.

FIG. 14 is a schematic view showing use of the FIG. 1 failsafe tourniquet with its pneumatic device in its initial closed pocketknife state as a windlass tourniquet for circumferentially applying limb occlusion pressure to a patient's limb.

### DETAILED DESCRIPTION OF DRAWINGS

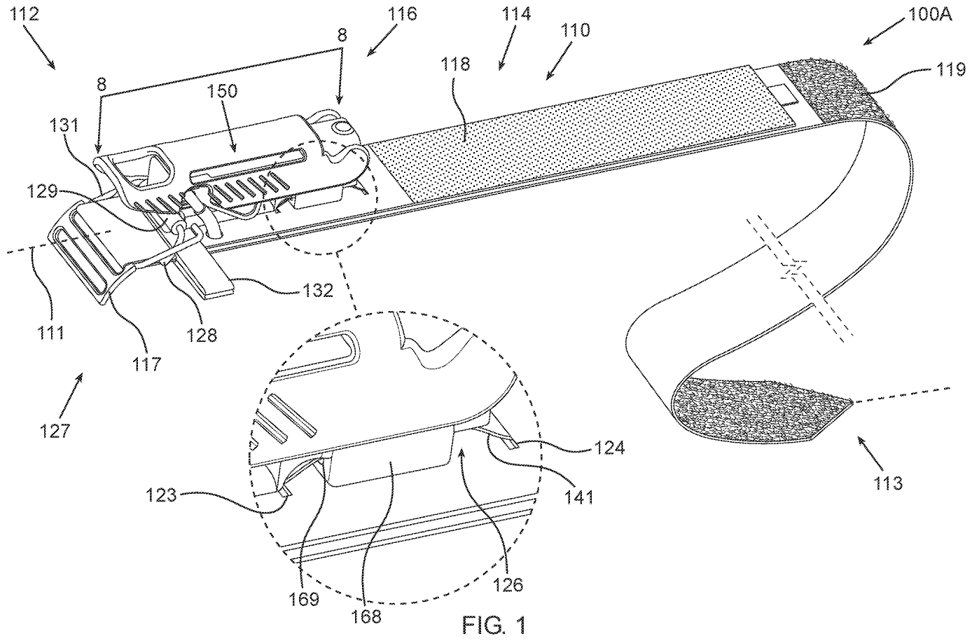

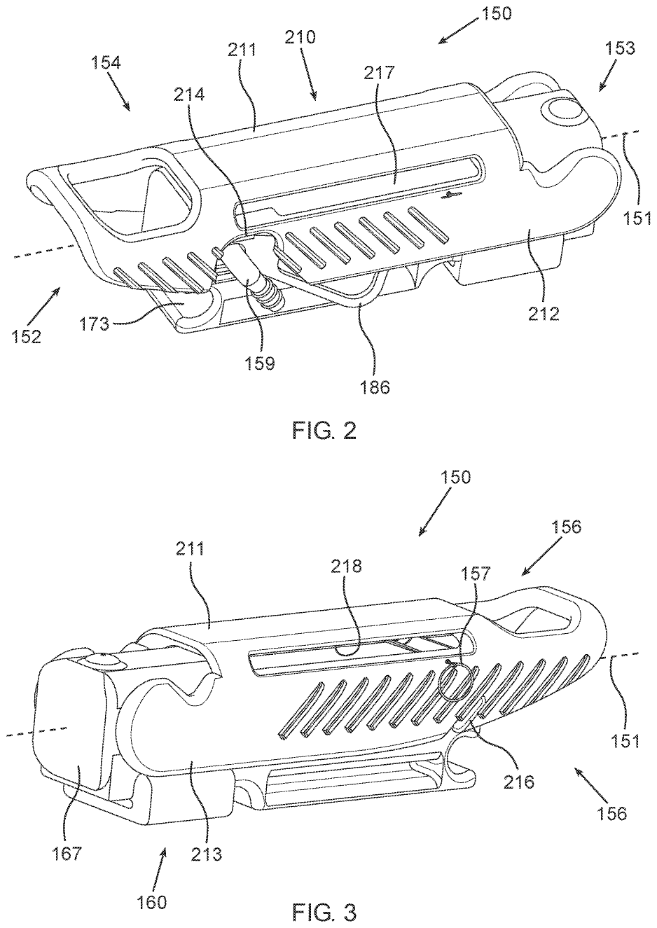

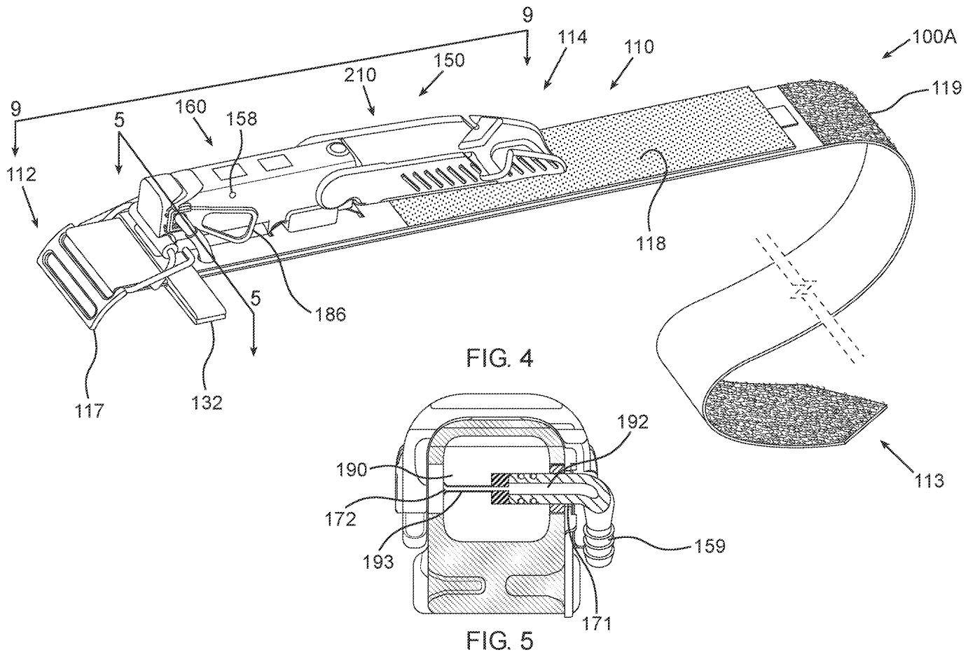

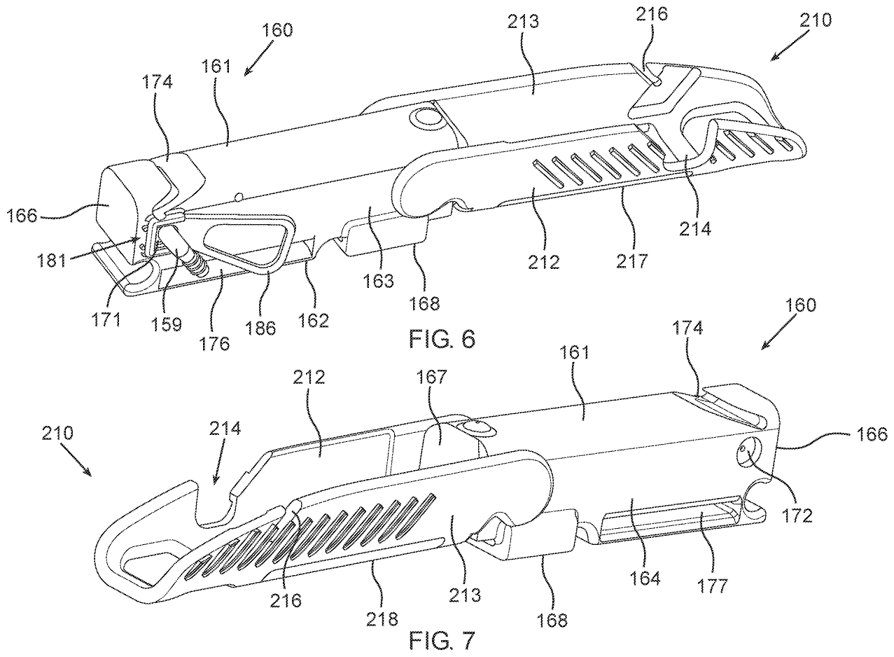

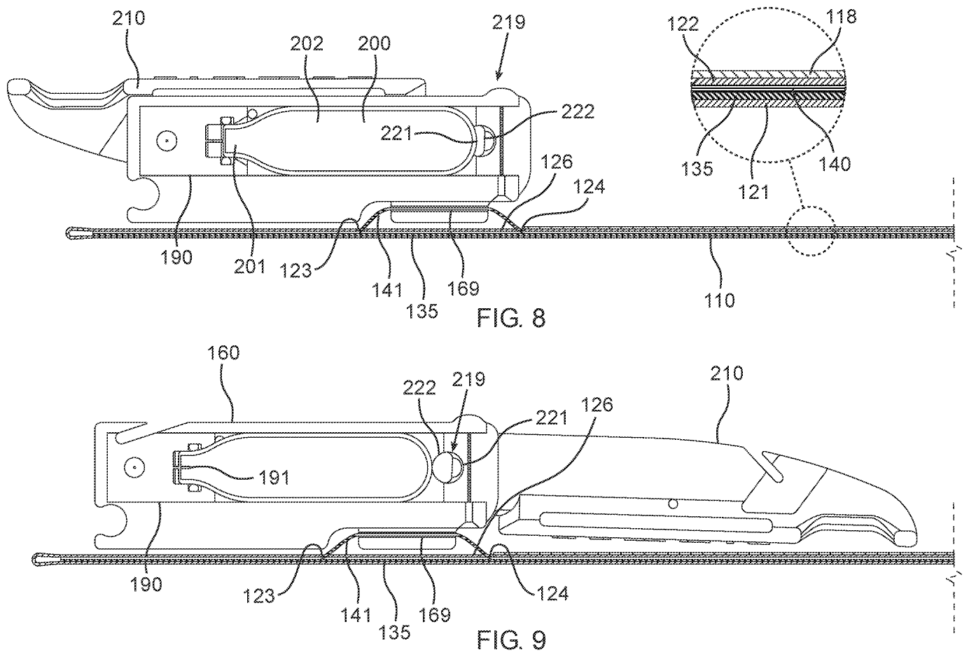

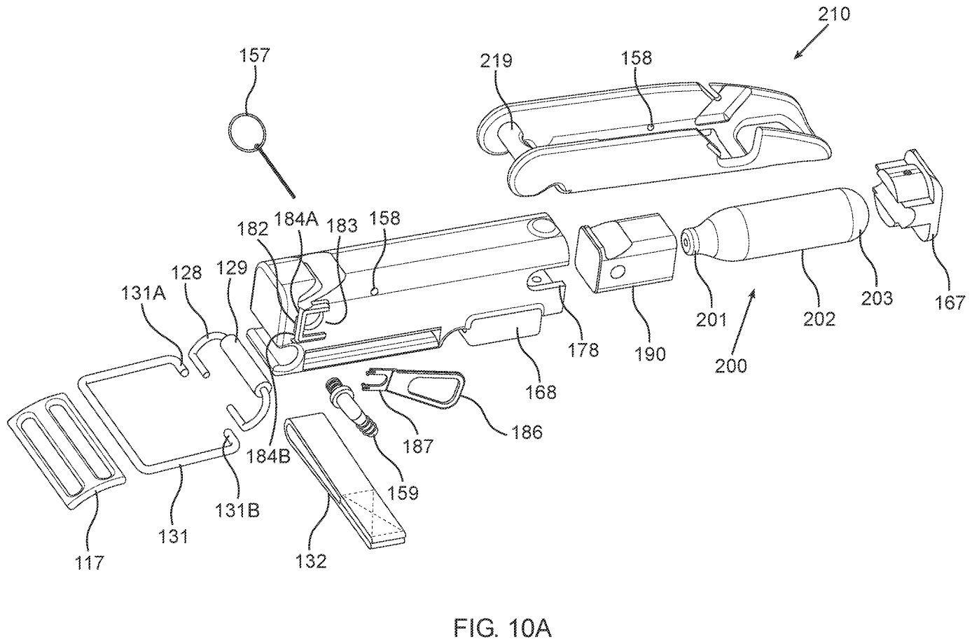

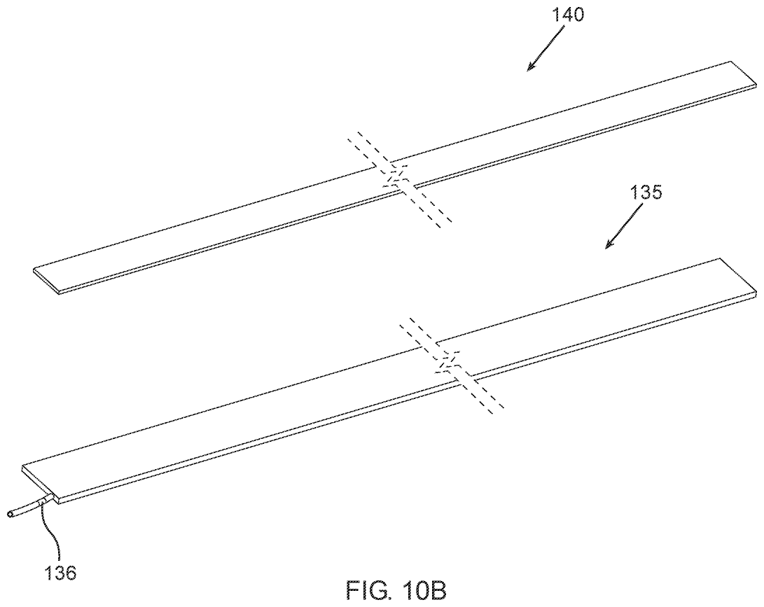

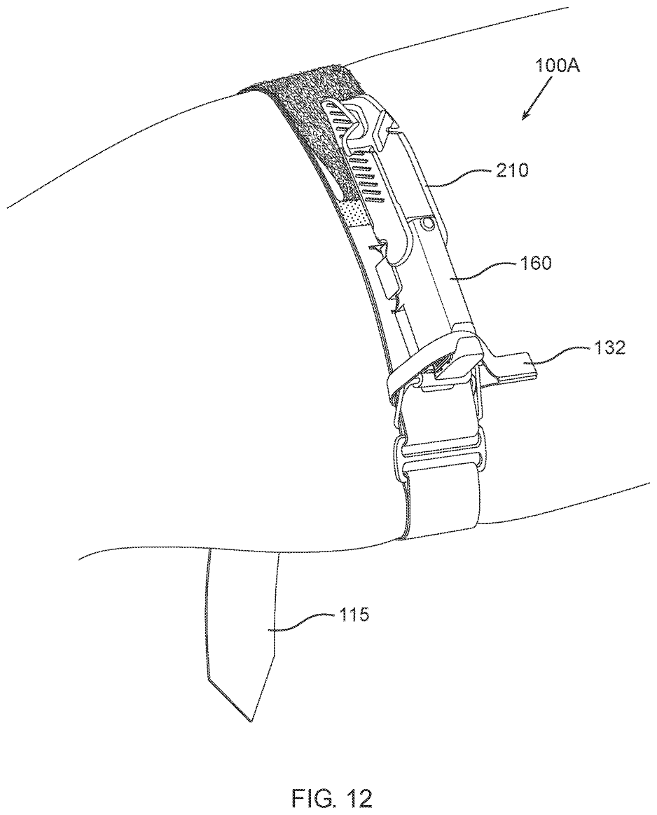

FIG. 1 to FIG. 10 show a failsafe tourniquet 100A for circumferentially applying limb occlusion pressure to a patient's limb. The failsafe tourniquet 100A includes an initially flat, elongated cuff 110 having a longitudinal cuff centerline 111, a cuff buckle end 112 and a cuff free end 113 opposite the cuff buckle end 112. Failsafe tourniquets 100A can be provided with cuffs 110 of different cuff lengths for applying to a patient's arm or a patient's leg ranging from small children to oversized adults. The cuff 110 has a cuff section 114 starting from the cuff buckle end 112 and partially extending towards the cuff free end 113 which is intended to encircle a patient's limb. The cuff 110 includes a fastening arrangement 116 for fastening the cuff section 114 around a patient's limb, thereby leaving a freely hanging cuff remaining section 115 (see FIG. 12 to FIG. 14). The fastening arrangement 116 includes a dual square loop buckle 117 at the cuff buckle end 112, a Velcro hook strip 118 mounted on the cuff 110 towards the cuff buckle end 112 and substantially co-extensive with the cuff section 114 and a Velcro loop strip 119 extending from the Velcro hook strip 118 towards the cuff free end 113 for fastening on the Velcro hook strip 118. The failsafe tourniquet 100A includes a pneumatic device 150 mounted on the cuff 110 for tightening the cuff 110 for circumferentially applying limb occlusion pressure to a patient's limb as described hereinbelow.

The cuff 110 has a dual ply construction including an underside ply 121 facing towards a patient's limb and a topside ply 122 overlying the underside ply 121 and facing away from a patient's limb. The Velcro hook strip 118 and the Velcro loop strip 119 are placed on the topside ply 122. The cuff 110 can optionally have additional plies, for example, for strengthening purposes. The topside ply 121 includes a pair of opposite spaced apart topside ply slits 123 and 124 transverse to the longitudinal cuff centerline 111 towards the cuff buckle end 112. The pair of opposite spaced apart topside ply slits 123 and 124 bound a topside ply section 126.

The cuff buckle end 112 includes a restraining arrangement 127 for assisting use of the failsafe tourniquet 100A in both its inflation mode of operation and its windlass mode of operation. The restraining arrangement 127 includes an inverted U-shaped frame 128 with a cylindrical restraining member 129, a U-shaped frame 131 with U-shaped frame free ends 131A and 131B, and an elastic safety strap 132.

The failsafe tourniquet 100A includes a strip balloon 135 deployed inside the cuff 110 between the underside ply 121 and the topside ply 122 starting at the cuff buckle end 112 and substantially co-extensive with the cuff section 114. The strip balloon 135 is configured to be inflated to a predetermined limb occlusion pressure. The strip balloon 135 preferably includes a one-way valve 136 for ensuring it remains inflated after inflation. The strip balloon 135 can be attached to the cuff 110 using conventional manufacturing techniques including inter alia gluing, stitching, ultrasonic welding, and the like.

The failsafe tourniquet 100A includes a windlass strap 140 integral with the cuff 110 and deployed inside the cuff 110 between the topside ply 122 and the strip balloon 135 starting at the cuff buckle end 112 and substantially co-extensive with the cuff section 114. The windlass strap 140 can be deployed in the cuff 110 using conventional manufacturing techniques including inter alia gluing, stitching, ultrasonic welding, and the like. The windlass strap 140 typically has the same length as the strip balloon 135. The windlass strap 140 underlies the topside ply 122 except for a windlass strap section 141 which exits from the topside ply 122 at the topside ply slit 123, freely overlies the topside ply section 126 for enabling assembly of the failsafe tourniquet 100A as described hereinbelow, and enters under the topside ply 122 at the topside ply slit 124.

The hand operated pneumatic device 150 has a longitudinal pneumatic device centerline 151 and is mounted on the windlass strap section 141 whereby it can be rotated in a clockwise or counterclockwise direction overlying the cuff 110 for use as a windlass tourniquet. The pneumatic device 150 has a pneumatic device leading end 152 adjacent the cuff buckle end 112 and a pneumatic device trailing end 153 remote from the cuff buckle end 112. The pneumatic device 150 includes a pneumatic device housing 154 having an open ended, hollow, generally parallelopiped base 160 and a generally elongated U-shape cross section handle 210 pivotal thereon about an about half turn rotation from an initial closed pocketknife state in which the handle 210 overlies the base 160 to a final open pocketknife state in which the handle 210 extends co-directional with the base 160. The pneumatic device 150 in its initial closed pocketknife state is initially co-directional with the cuff 110.

The pneumatic device 150 includes a safety pin arrangement 156 for securing the handle 210 to the base 160 to prevent opening the pneumatic device 150 without releasing the safety pin arrangement 156. The safety pin arrangement 156 includes a finger released safety pin 157 slidingly inserted into a throughgoing safety pin bore 158 traversing the base 160 and the handle 210.

The base 160 includes a base top surface 161, a base bottom surface 162 opposite the base top surface 161, a base front surface 163, a base rear surface 164 opposite the base front surface 163, a base leading end face 166 and a removable base trailing cap 167 opposite the base leading end face 166. The base 160 includes an integrally formed windlass strap support 168 under the base bottom surface 162 towards the base trailing cap 167, thereby bounding a windlass strap channel 169 through which the windlass strap section 141 is threaded therethrough. The base front surface 163 and the base rear surface 164 respectively include a base front surface port 171 towards the base leading end surface 166 and a base rear surface port 172 opposite the base front surface port 171. The base rear surface port 172 can be optionally provided with a pressure regulator valve. Alternatively, the pressure regulator valve can be provided in the base leading end face 166. The base top surface 161 includes a DATE label and a TIME label for enabling a user to write the failsafe tourniquet 100A's date and time of use in the failsafe tourniquet 100A's final open pocketknife state.

The base leading end surface 166 includes a base leading end surface groove 173 for removably receiving the cylindrical restraining member 129 in the failsafe tourniquet 100A's initial closed pocketknife state and final open pocketknife state. The cylindrical restraining member 129 restrains the pneumatic device 150 co-directional with the cuff 110 and in close proximity with the cuff 110 during hand opening the pneumatic device 150 from its initial closed pocketknife state to its final open pocketknife state. The cylindrical restraining member 129 is removed from the base leading end surface groove 173 for enabling winding of the pneumatic device 150 as a windlass rod.

The base top surface 161 includes a base top surface groove 174 towards base leading end face 166 for receiving the elastic safety strap 132 in the failsafe tourniquet 100A's final open pocketknife state. The base front surface 163 and the base rear surface 164 respectively include a base front surface groove 176 and a base rear surface groove 177 opposite the base front surface groove 176 towards the base bottom surface 162 and the base leading end surface 166 for receiving the U-shaped frame free ends 131A and 131B in the failsafe tourniquet 100A's final open pocketknife state.

The base front surface 163 and the base rear surface 164 respectively include a base front surface cutout 178 and a base rear surface cutout 179 (not shown) opposite the base front surface cutout 178 towards the base trailing cap 167.

The base 160 accommodates a box-shaped gas cylinder housing 190 on sliding insertion thereinto and subsequent securing therein by the base trailing cap 167. The gas cylinder housing 190 is configured to hold an initial sealed compressed gas cylinder 200 having a gas cylinder boss 201 and a gas cylinder body 202 with a gas cylinder trailing end 203. The gas cylinder housing 190 sealingly grips the gas cylinder boss 201 and includes a puncturing member 191 for puncturing the gas cylinder boss 201 on sliding displacement of the gas cylinder 200 towards the puncturing member 191. The gas cylinder housing 190 includes a gas cylinder housing front channel 192 in flow communication with the base front surface port 171 and a gas cylinder housing rear channel 193 in flow communication with the base rear surface port 172. The gas cylinder housing front channel 192 has a wider diameter than the gas cylinder housing rear channel 193 for providing less gas flow resistance. The gas cylinder 200 contains more gas contents than required to inflate the strip balloon 135 to its predetermined limb occlusion pressure such that the pneumatic device 150 necessarily vents excess gas contents during use. The excess gas to be released depends on a limb's circumference at which the failsafe tourniquet 100A is being applied.

The pneumatic device 150 includes a gas hose 159 for flow communication between the base front surface port 171 and the strip balloon 135. The base front surface 163 includes a square U-shaped restraining member 181 towards the pneumatic device leading end 152 co-directional with the longitudinal pneumatic device centerline 151. The square U-shaped restraining member 181 includes a square U-shaped restraining member crosspiece 182 towards the pneumatic device leading end 152 and a square U-shaped restraining member opening 183 towards the pneumatic device trailing end 153. The square U-shaped restraining member crosspiece 182 includes a spaced apart pair of restraining apertures 184A and 184B. The pneumatic device 150 includes a quick release retainer clip 186 with a bifurcated quick release retainer clip leading end 187 for sliding insertion into the spaced apart pair of restraining apertures 184A and 184B. The quick releaser retainer clip 186 traps the gas hose 159 in place. Sliding release of the quick release retainer clip 186 enables detachment of the gas hose 159 from the base front surface port 171.

The handle 210 includes a handle crosspiece 211, a handle front surface 212 and a handle rear surface 213 opposite the handle front surface 212. The handle front surface 212 and the handle rear surface 213 respectively include an inclined handle front surface cutout 214 and an inclined handle rear surface cutout 216 for receiving the elastic safety strap 132 in the failsafe tourniquet 100A's final open pocketknife state. The handle front surface 212 and the handle rear surface 213 respectively include an elongated handle front surface groove 217 and an elongated handle rear surface groove 218 opposite the handle front surface groove 217 towards the handle crosspiece 212 for receiving the U-shaped frame free ends 131A and 131B in the failsafe tourniquet 100A's final open pocketknife state and after the pneumatic device 150 has been wound by one or more half turns for acting as a windlass tourniquet.

The handle 210 includes an axle 219 rotatably mounted in the base front surface cutout 178 and the base rear surface cutout 179. The axle 219 includes an axle leading surface 221 contacting the gas cylinder trailing end 203 in the failsafe tourniquet 100A's initial closed pocketknife state and an axle trailing surface 222 contacting the gas cylinder trailing end 203 in the failsafe tourniquet 100A's final open pocketknife state. The axle leading surface 221 is dimensioned such that the gas cylinder 200 is snugly deployed between the puncturing member 191 and the axle leading surface 221 without the gas cylinder 200 being punctured. The axle trailing surface 222 is dimensioned to slidingly displace the gas cylinder 200 towards the puncturing member 191 for puncturing the gas cylinder 200.

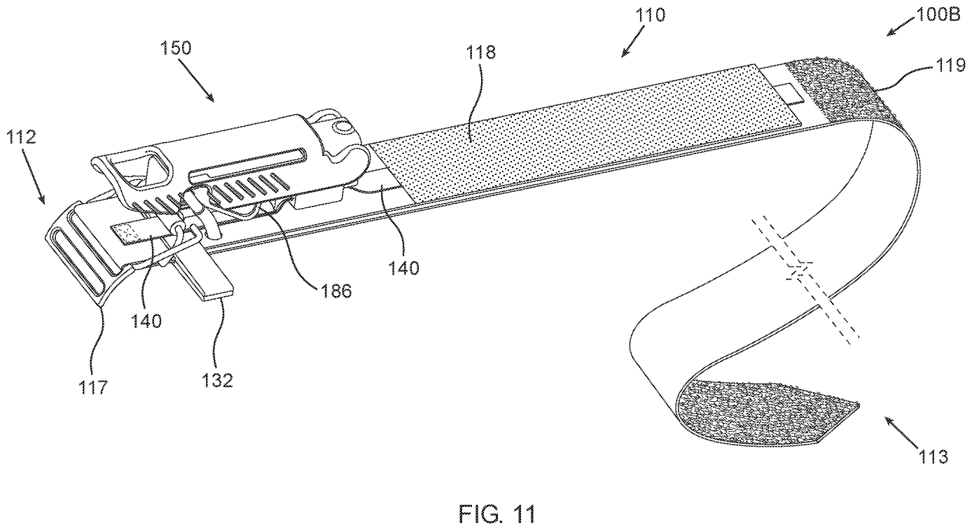

FIG. 11 shows a failsafe tourniquet 100B having a similar construction and operation as the failsafe tourniquet 100A and therefore similar parts are likewise numbered. The failsafe tourniquet 100B differs from the failsafe tourniquet 100A insofar that the windlass strap is formed integral with the cuff 110 and is deployed overlying the topside ply and beneath the Velcro hook strip. The pneumatic device 150 is mounted on the windlass strap section in the same manner as the failsafe tourniquet 100A.

FIG. 12 to FIG. 14 show use of the failsafe tourniquet 100A. A user typically prefers to inflate the failsafe tourniquet rather than using the failsafe tourniquet as a windlass tourniquet because inflating the failsafe tourniquet requires less user actions and is accordingly quicker than using the windlass tourniquet as a windlass tourniquet. However, in an instance, a user is aware that the failsafe tourniquet has to apply less or more limb occlusion pressure than the strip balloon's predetermined limb occlusion pressure, the user operates the failsafe tourniquet as a windlass tourniquet. Additionally, in an instance, a user inflates the failsafe tourniquet but the failsafe tourniquet fails to sufficiently limit or stop a patient's bleeding, the user can use the failsafe tourniquet as a windlass tourniquet for applying greater limb occlusion pressure than the strip balloon's predetermined limb occlusion pressure. Also, in an instance, a user intends to inflate the failsafe tourniquet but its strip balloon doesn't inflate or ruptures, the user can use the failsafe tourniquet as a windlass tourniquet.

FIG. 12 shows a user threading the cuff free end through the buckle for encircling a patient's limb and fastening the cuff therearound. The user removes the safety pin and opens the pneumatic device from its initial closed pocketknife state to its final open pocketknife state by rotating the handle by a half turn. Rotation of the handles punctures the gas cylinder for inflating the cuff to the strip balloon's predetermined limb occlusion pressure. Excess compressed gas is vented via the base rear surface port. The user confirms the failsafe tourniquet is applying sufficient limb occlusion pressure to sufficiently limit bleeding. The user places the elastic safety strap on the base top surface groove. The user writes the date on the DATE label and the time on the TIME label.

In an instance that operating the pneumatic device does not inflate the cuff or the cuff applies insufficient limb occlusion pressure for limiting bleeding, the user prepares the failsafe tourniquet for use as a windlass tourniquet. The user removes the restraining member from the base leading end surface groove and detaches the quick release retainer clip. The user detaches the gas hose from the base front surface port. The user winds the pneumatic device in its final open pocketknife state around the windlass strap section for tightening the cuff around the patient's limb for circumferentially applying limb occlusion pressure to stop bleeding.

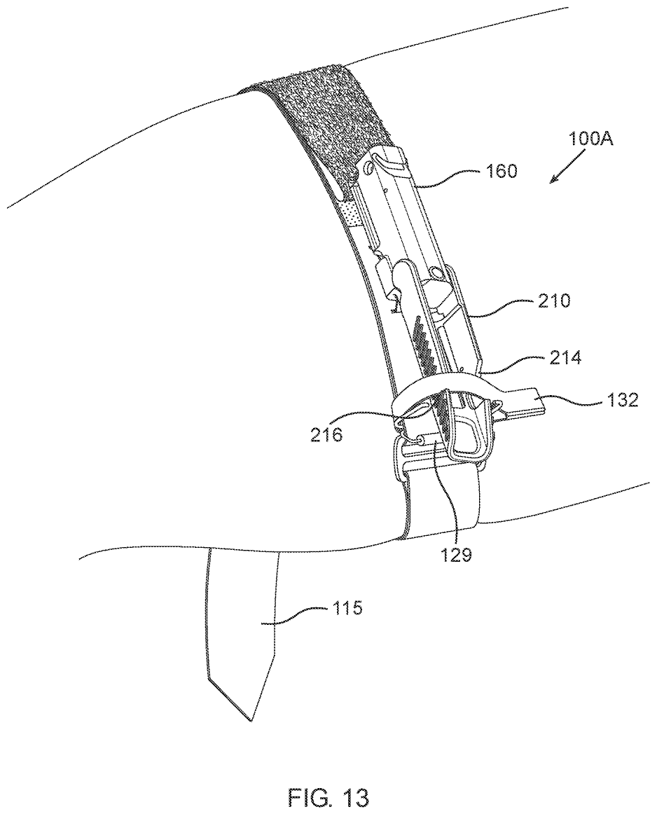

On stopping bleeding, in the case that the user rotates the pneumatic device an odd number of half turns, the handle is directed towards the cuff buckle end and the base is directed towards the cuff free end (see FIG. 13). The user places the elastic safety strap across the handle front surface cutout and handle rear surface cutout and correspondingly inserts the U-shaped frame free ends and into the handle front surface groove and the handle rear surface groove.

On stopping bleeding, in the case that the user rotates the pneumatic device an even number of half turns, the base is directed towards the cuff buckle end and the handle is directed towards the cuff free end. The user places the places the elastic safety strap on the base top surface groove and correspondingly inserts the U-shaped frame free ends into the base front surface groove and the base rear surface groove.

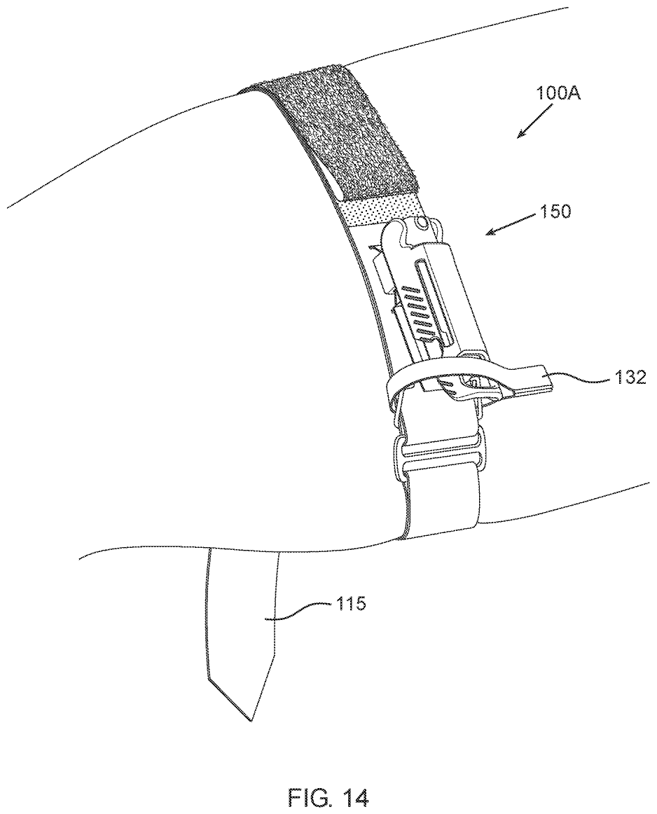

FIG. 14 shows a user threading the cuff free end through the buckle for encircling a patient's limb and fastening the Velcro loop strip on the Velcro hook strip. The user does not operate the pneumatic device but rather prepares the failsafe tourniquet for use as a windlass tourniquet. The user removes the restraining member from the base leading end surface groove and detaches the quick release retainer clip for detaching the gas hose from the base front surface port. The user winds the pneumatic device in its initial closed pocketknife state around the windlass strap section for tightening the cuff around the patient's limb for circumferentially applying limb occlusion pressure to stop bleeding. On stopping bleeding, in the case that the user rotates the pneumatic device an even number of half turns, the base is directed towards the cuff buckle end and the handle is directed towards the cuff free end. The user places the elastic safety strap on the base top surface groove and correspondingly inserts the U-shaped frame free ends into the base front surface groove and the base rear surface groove.

While the invention has been described with respect to a limited number of embodiments, it will be appreciated that many variations, modifications, and other applications of the invention can be made within the scope of the appended claims.

1. A failsafe tourniquet for circumferentially applying a limb occlusion pressure to a patient's limb, the failsafe tourniquet comprising:
a) an initially flat, elongated cuff including a longitudinal cuff centerline and having:
i) a cuff buckle end,
ii) a cuff free end opposite the cuff buckle end,
iii) a cuff section starting from the cuff buckle and partially extending towards the cuff free end and intended to be encircled around the patient's limb,
iv) a fastening arrangement for fastening the cuff section encircled around the patient's limb,
v) an underside ply for intimate contact with a patient's limb, and
vi) a topside ply overlying the underside ply;
b) an initially uninflated strip balloon deployed between the underside ply and the topside ply, the initially uninflated strip balloon starting at the cuff buckle end and substantially co-extension with the cuff section, the strip balloon being configured for being inflated to a predetermined limb occlusion pressure;
c) a windlass strap integral with the cuff, the windlass strap starting at the cuff buckle end and substantially co-extensive with the cuff section, the windlass strap including a windlass strap section freely overlying the topside ply and deployed towards the cuff buckle end; and
d) a hand operated pneumatic device mounted on the windlass strap section towards the cuff buckle end, the hand operated pneumatic device including an initially sealed gas cylinder containing gas contents for selectively inflating the initially uninflated strip balloon,
the pneumatic device being configured to tighten the cuff section encircled around the patient's limb either by a user operating the pneumatic device for puncturing the initially sealed gas cylinder for inflating the cuff section for applying the predetermined limb occlusion pressure or winding the pneumatic device around the windlass strap section in a windlass rod-like manner.

2. The failsafe tourniquet according to claim 1 wherein the pneumatic device includes a base and a handle pivotal on the base in a pocketknife manner from an initial closed pocketknife state to a final open pocketknife state on an about half turn rotation of the handle relative to the base.

3. The failsafe tourniquet according to claim 2 wherein the pneumatic device includes a safety pin arrangement including a finger-pulled safety pin slidingly inserted in a safety pin throughgoing bore traversing the base and the handle for securing the handle to the base to prevent opening the pneumatic device from its initial closed pocketknife state to its final open pocketknife state without releasing the safety pin arrangement.

4. The failsafe tourniquet according to claim 2 wherein the base includes a puncturing member for puncturing the initially sealed gas cylinder and the handle includes an axle for slidingly displacing the initially sealed gas cylinder towards the puncturing member on being rotated from its initial closed pocketknife state to its final open pocketknife state for puncturing the gas cylinder in its final open pocketknife state.

5. The failsafe tourniquet according to claim 1 wherein the pneumatic device includes a gas hose extending to the strip balloon and further comprising a quick release retainer clip mounted on the pneumatic device for retaining the gas hose whereupon, in the final open pocketknife state, detaching the quick release retainer clip from the pneumatic device enables detachment of the gas hose from the pneumatic device thereby enabling winding the pneumatic device as a windlass rod.

6. The failsafe tourniquet according claim 1 wherein the strip balloon includes a one-way valve for enabling inflation of the strip balloon to a predetermined limb occlusion pressure and the pneumatic device includes a port for releasing excess gas after inflating the strip balloon to its predetermined limb occlusion pressure.

7. The failsafe tourniquet according to claim 1 wherein the topside ply includes an opposite pair of transverse topside ply slits towards the cuff buckle end bounding a topside ply section and the windlass strap underlies the topside ply except for the windlass strap section which exits from the topside ply at a first topside ply slit of the opposite pair of topside ply transverse slits, overlies the topside ply section and enters under the topside ply at a second topside ply slit of the opposite pair of transverse topside ply slits.

8. The failsafe tourniquet according to claim 1 wherein the windlass strap overlies the topside ply.
